(19) United States
(12) Patent Application Publication
(10) Pub. No.: US 2019/0175111 A1
(43) Pub. Date: Jun. 13, 2019

(54) DEVICES AND METHODS FOR ATRIAL MAPPING, SENSING AND TREATING CARDIAC ARRHYTHMIA

(71) Applicant: 4C Medical Technologies, Inc., Brooklyn Park, MN (US)

(72) Inventors: Philippe Genereux, New York, NY (US); Robert Kipperman, Morristown, NJ (US); Saravana B. Kumar, Minnetonka, MN (US)

(21) Appl. No.: 16/207,778

(22) Filed: Dec. 3, 2018

Related U.S. Application Data
(60) Provisional application No. 62/594,089, filed on Dec. 4, 2017.

(51) Int. Cl. A61B 5/00; A61N 1/39; A61N 1/362

(52) U.S. Cl. CPC A61B 5/6862; A61B 5/686; A61B 5/0464; A61N 1/3622; A61N 1/3956

(57) ABSTRACT

The present invention comprises devices, systems and methods for providing an implanted device within a patient's atrium, wherein the implanted device comprises one or more sensors and one or more electrodes associated with the implanted device. The sensors may be used in various embodiments to monitor for arrhythmia, and in some embodiments may map the arrhythmia by detecting, inter alia, flutter and/or defibrillation, and the electrodes may be adapted to respond to any detected arrhythmia with electrostimulation including, inter alia, cardioversion and/or ablation.

### CROSS-REFERENCE TO RELATED APPLICATIONS

This application claims the benefit of U.S. Provisional Application Ser. No. 62/594,089, filed Dec. 4, 2017 and entitled APPLICATION OF COMPLIANT STENTING FOR ATRIAL MAPPING, SENSING, PACING & ABLATION CAPABILITIES, the entirety of which is hereby incorporated by reference.

### STATEMENT REGARDING FEDERALLY SPONSORED RESEARCH OR DEVELOPMENT

Not applicable

### FIELD OF THE INVENTION

The invention relates to devices, methods and systems for providing continuous or transient monitoring ad mapping capability of arrhythmia and for providing electrostimulation to the patient's atrial chamber.

### BACKGROUND OF THE INVENTION

Atrial fibrillation or flutter are frequent symptoms of arrhythmia. Continuous monitoring, immediate arrhythmia identification, exact mapping location, and prompt, focused treatment could lead to improvement in prognostic for patient suffering from arrhythmia.

It would be advantageous to provide sensors and/or electrodes on a compliant stent implanted within a patient's left or right atrium. Such an arrangement would enable continuous and/or transient monitoring and mapping of a patient's arrhythmia. Further, such an arrangement may enable rapid pacing to terminate or cardiovert any cardiac arrhythmia detected by the sensors incorporated into or on the implanted stent.

The present invention addresses these, inter alia, issues.

### BRIEF DESCRIPTION OF THE SEVERAL VIEWS OF THE DRAWINGS

FIG. 1 illustrates a side view of one embodiment of the present invention.

FIG. 2 illustrates a side view of one embodiment of the present invention.

### BRIEF SUMMARY OF THE INVENTION

The present invention comprises devices, systems and methods for providing an implanted device within a patient's atrium, wherein the implanted device comprises one or more sensors and one or more electrodes associated with the implanted device. The sensors may be used in various embodiments to monitor for arrhythmia, and in some embodiments may map the arrhythmia by detecting, inter alia, flutter and/or defibrillation, and the electrodes may be adapted to respond to any detected arrhythmia with electrostimulation including, inter alia, cardioversion and/or ablation.

### DETAILED DESCRIPTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

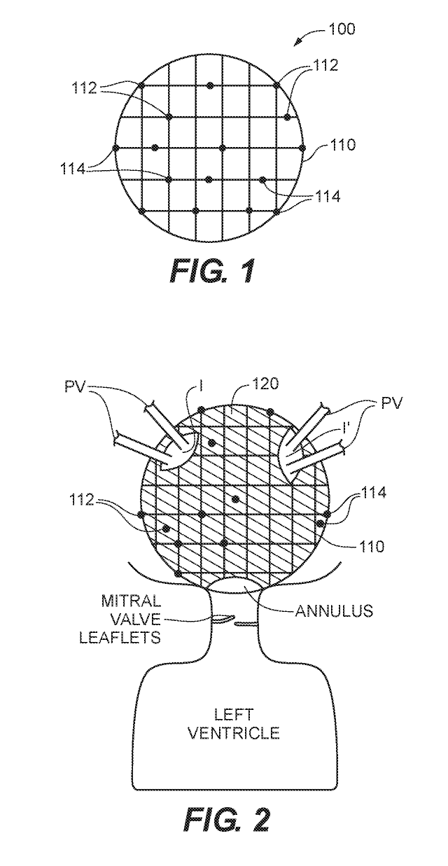

The various embodiments of the present invention comprise generally a prosthetic implant located within a patient's left or right atrium. Generally, various embodiments of the invention comprises one or more sensors and one or more electrodes mounted on, or integrated into, the prosthetic implant, e.g., on or within an expandable stent. An exemplary implantable device 100 comprising a spherical expandable stent frame 110 is illustrated in FIG. 1. There, possible mounting locations, or integration locations are illustrated at the intersection of struts for sensors 112 and electrodes 114. These mounting or integration locations are merely exemplary. Therefore, the sensor(s) 112 and electrode(s) 114 may be located at any location along the expandable stent and/or as discussed below on or in a coating or covering of the expandable stent.

Though FIG. 1 provides the device 100 comprising both sensors 112 and electrodes 114, various embodiments may only comprise sensors 112 or only electrodes 114.

Expandable stents 110 are well known in the art. Further, stenting within the left or right atrium may be achieved using known stenting techniques, including but not limited to self-expandable stents or balloon expandable (or any other stent expansion mechanism). The stent frame 110, as expanded within the atria, may comprise a shape that expands to at least a portion of the atrial chamber space and/or may comprise spherical, oval, cylindrical/or other shapes, so long as the expanded stent frame 110 is at least partially apposed, and anchored, to the tissue of the atrium.

The stent frame 110 of the present invention may comprise a flexible and compliant shape memory alloy, such as but not restricted to nitinol. The design of the stent frame 110 could be an open cell strut design and/or with its inside and/or outside surfaces at least partially covered with a mesh or fabric, or coated with polytetrafluoroethylene (PTFE) to favor rapid endothelialization. This covering or coating is shown in FIG. 2 at element 120.

In preferred embodiments, stent frame 110 is partially, but mostly, covered and comprises uncovered areas to allow pulmonary vein flow and mitral valve inflow/outflow. Generally, the coating or covering 120 will not cover or coat inflow I, I′ and outflow O regions of the implanted device that correspond with blood inflow into the atrium and blood outflow from the atrium. Thus, in the case of the implanted device within the left atrium, the expanded stent may be covered, with the exception of regions corresponding with the pulmonary vein (PV) entry points within the left atrium, and the location of the mitral annulus and related channel leading to the left ventricle. The expanded stent 110 may comprise an open cell or strut construction throughout, with the inflow I, I′ and outflow O regions uncovered. Alternatively, as shown in FIG. 2, the mitral opening at outflow region O in stent frame 100 may comprise an opening in the expandable stent, i.e, no stent frame struts, cells or coating or covering 120 are present in that outflow region O of the stent frame 110. Similarly, inflow regions I, I′ at the pulmonary vein inflow points PV may comprise an opening in the expandable stent at these inflow locations where there are no struts, cells or covering.

The stent frame 110 may comprise integrated sensors 112 and/or electrodes 114 and/or additional implanted sensors 112 and/or electrodes 114 mounted on the stent frame 110. Similarly, the mesh or coating 120 covering the stent frame 110 may have continuous or transient electrophysiological monitoring capability, mapping, sensing, pacing, shocking, or ablation capability, either intrinsically, or externally via interaction with another device. Generally, sensors 112 and/or electrodes 114 are associated (either mounted on or integrated with) the stent frame 110 prior to delivery, expansion and anchored implant within the patient's atrium. Alternatively, in other embodiments, one or more sensors 112 and/or electrodes 114 may be delivered and connected or otherwise associated with the implanted device 100 after the stent frame 110 has been expanded for implantation

In the case where implanted device 100 is implanted in the left atrium, the coating or covering 120 of the at least partially covered stent frame 110 may further function to exclude/occlude the left atrial appendage (LAA) after successful endothelialization of the implant to prevent clotting therein. In this embodiment, an unbroken coating or covering 120 covering the outer portion of the stent frame in the location of the LAA is preferred to ensure coverage, and therefore closure or blockage, of the LAA upon implantation and subsequent endotheliazation. A preferred coating for this purpose comprises PTFE, though equivalent coating or covering materials will readily present themselves to the skilled artisan, each of which is within the scope of the present invention.

The implanted device 100 may be delivered using transcatheter or surgical techniques. The delivery access may include transfemoral, transaortic, transapical, transatrial, transjugular or similar pathways.

The implanted device 100 may include a self-expandable stent, a balloon expandable stent, and/or a surgically placed device that is conformable to the atrial anatomy. Stent frame 100 conformation to the atrium shaping and/or apposition to the inner surface(s) of the atrium may be achieved using any one of several known techniques.

Self-expanding stent frames 110 may be made using diamond shaped cell patterns using laser cut or similarly processed nitinol tubing. The geometry of the stents may be tailored to individual patient anatomies and/or generic shapes to conform to expected patient anatomies. The radial forces of the stents are tailored to the compliance of the atrial anatomy using a combination of tubing properties, processing, and geometrical dimensions to achieve appropriate forces.

Self-expanding stent frames 110 may also be similarly processed from nitinol wires, braids, and/or sheet materials to a similar combination of radial forces, geometry and processing parameters to achieve requiring compliance to provide sufficient contact with the interior surfaces of the relevant atrial chamber for anchoring, flexibility of motion to accommodate the natural pulsatile/contractile movements of the atrial chamber and to enable the various sensing and treating mechanisms described herein.

Balloon expandable stent frames 110 may be constructed using typically used medical device materials (e.g. stainless steel, cobalt chromium etc.). Compliance of the stents are tailored using geometry and processing to achieve necessary force responses on expansion using a balloon or similar techniques.

The one or more sensor/electrodes 112, 114 may be attached to the stent frame 110 using mechanical attachment features (e.g. swage, weld, locks), adhesive and/or other equivalent methods. Examples of sensors 112 or electrodes 114 that may be secured to the stent frame 110 are discussed below. Alternatively, sensors 112 and/or electrodes 114 may be integrated into the stent frame 110 or other elements of the expandable stent, e.g., the inner and/or outer coating or covering 120 when present.

One form of sensor 112 comprises one or more pressure sensors may be attached to specific locations of the implanted device as discussed above to monitor cardiac pressure as a function of cardiac cycle, disease state, and similar criteria to obtain patient specific responses. The pressure sensors may function by converting changes to capacitance, inductance, flow rate or similar characteristics into a reliable pressure mapping at locations within the atrium and, therefore, the term pressure sensor is defined herein as any sensor that is adapted to measure an electrical characteristic that may be converted either directly or indirectly into a pressure map. The information may also be transmitted wirelessly to a receiver for data monitoring and analysis.

The one or more pressure sensors may be further, or alternatively, attached next to, or proximate, the pulmonary veins or arteries in the inflow regions I, I′ on expandable stent 110 to measure and map flow and pressure responses as a function of cardiac cycle, disease state or similar characteristics.

One or more pressure sensors may further be provided at outflow region O of the relevant atrium, and implanted device's expandable stent frame 110, to monitor and map cardiac activity, including pressure and fluid flow characteristics, within the atrium.

Another form of sensor 112 may comprise flow sensors attached to, or integrated with, stent frame 110 and/or coating or covering 120 to monitor blood flow volume and velocity at different locations within the atrium.

The prosthetic implant may be attached separately and used to transmit electrical (electrodes) or mechanical stimulators (e.g. ablation, cryoablation, pulses etc.) using transcatheter techniques to conduct to specific locations of the atrium.

A separate electrode 112 or electrodes 112 may be attached to the implant after deployment into the atrium to transmit electrical or mechanical impetus to the anatomy. Such impetus may be used for pulmonary vein ablation or left atrium ablation for the treatment of atrial fibrillation.

Electric current—both DC and AC—may be transmitted to the sensors 112 and/or electrodes 114 and/or stent frame 110 as desired to achieve the therapeutic function described herein. The implanted device 100 may be self-powered or powered by a wireless external generator implanted under the skin.

Mechanical and/or thermal stimulators may be attached to the implant either during or after deployment to transmit heat, cold or other stimulants to selective ablate or treat specific locations within the atrial anatomy.

The prosthetic implanted device 100 may be placed either in the left atrium or the right atrium with appropriate sensors/electrodes, 112, 114.

The implanted device 100 may comprise complete or partial bioabsorption capability, with or without preservation of the sensors, electrodes to perform ablation, cardio stimulation, or cardioversion.

Generally, the therapeutic processes that may be achieved using the invention described herein follow.

1) Any continuous or transient monitoring and mapping capability of arrhythmia within the atrium using the one or more sensors 112.

2) Rapid pacing capacity (rapid burst) of the electrode(s) 114 to terminate or cardioverse any cardiac arrhythmia detected by the sensor(s) 112.

3) Any arrhythmia cardioversion or ablation capacity using the implanted device 100 as a system to perform ablation, either invasively (endovascular, surgical) or transcutaneous (i.e vest, patch) or to reflect any type of energy originating from outside the body source.

4) Any drug eluting capability from the implanted device 100, either continuously delivered, transiently delivered, or with delivery triggered by the detection of data captured by the sensor(s) 112.

5) Stent frame 110 may be used as a docking station for any adjunctive therapy, including but not restricted to left atrial appendage occlusion, inter-atrial shunt valve, ring, or any other devices to maintain left to right shunt. Moreover, sensor(s) 112 and/or electrode(s) 114 may be deployed to the implanted device 100. Without limitation, one exemplary electrode(s) 114 may be added to the implanted device 100 in the area of one or more pulmonary veins PV for purposes of ablating material therein.

6) Preservation of atrial kick and contractility with prosthetic implanted device 100 by electrostimulation applied by electrode(s) 114, in some embodiments in combination with sensor(s) 112, or any other devices with sensing or pacing capability incorporated within the frame or any other structure of the implanted device 100.

The present invention should not be considered limited to the particular examples described above, but rather should be understood to cover all aspects of the invention. Various modifications, equivalent processes, as well as numerous structures to which the present invention may be applicable will be readily apparent to those of skill in the art to which the present invention is directed upon review of the present invention.

1. An implanted device for mapping of arrhythmia within a patient's atrium, comprising:
an expandable stent frame adapted to expand to engage tissue within the atrium to anchor within the atrium;
at least one sensor associated with the implanted device;
at least one electrode associated with the implanted device;
a power source associated with the implanted device adapted for powering the at least one sensor and at least one electrode.

2. The implanted device of claim 1, wherein the at least one sensor is configured to detect cardiac arrhythmia.

3. The implanted device of claim 2, further comprising the at least one sensor configured to map the location of the detected cardiac arrhythmia.

4. The implanted device of claim 3, wherein the at least one electrode is configured to terminate or cardiovert the detected arrhythmia.

5. The implanted device of claim 4, wherein the at least one electrode is configured to rapidly actuate to terminate or cardiovert the detected arrhythmia.

6. The implanted device of claim 5, wherein the at least one electrode is configured to ablate a region of the patient's atrium.

7. The implanted device of claim 4, wherein the at least one electrode is actuated to target a specific location within the patient's atrium corresponding to the mapped location of the detected cardiac arrhythmia.

8. The implanted device of claim 4, wherein the at least one electrode is configured to ablate the specific target location within the patient's atrium.

9. The implanted device of claim 2, wherein the at least one sensor monitors either continuously or discontinuously for cardiac arrhythmia.

10. The implanted device of claim 1, wherein at least a portion of the expandable stent frame is coated with a coating or covered with a covering.

11. The implanted device of claim 5, wherein an inflow and an outflow region of the expandable stent frame, corresponding with an inflow and outflow region of the atrium, is uncovered.

12. The implanted device of claim 11, wherein the coating or covering comprises PTFE.

13. The implanted device of claim 10, wherein the implanted device is implanted within the patient's left atrium and wherein the coating or covering covers over the patient's left atrial appendage.

14. The implanted device of claim 1, wherein at least part of the implanted device is biosorbable.

15. The implanted device of claim 1, wherein the at least one sensor and/or at least one electrode is mounted on the expandable stent frame.

16. The implanted device of claim 1, wherein the at least one sensor and/or at least one electrode is integrated with the expandable stent frame.

17. The implanted device of claim 1, wherein the implanted device is further adapted to preserve atrial kick and contractility of the atrium.

18. The implanted device of claim 17, further comprising the at least one electrode configured to provide electro-stimulation to the atrium.

19. The implanted device of claim 1, wherein the expandable stent comprises a sphere when expanded and not deformed.

20. The implanted device of claim 10, wherein the at least one sensor and/or at least one electrode is mounted to or integrated with the coating or covering.
